(19) United States
(12) Patent Application Publication
(10) Pub. No.: US 2011/0038797 A1
(43) Pub. Date: Feb. 17, 2011

(54) METHOD FOR DETECTING INFLAMMATORY DISORDERS OF THE CENTRAL NERVOUS SYSTEM

(75) Inventors: Robyn Klein, St. Louis, MO (US); Joshua Rubin, St. Louis, MO (US)

Correspondence Address: POLSINELLI SHUGHART PC, 100 SOUTH FOURTH STREET, SUITE 1000, SAINT LOUIS, MO, 63102-1825, US

(73) Assignee: THE WASHINGTON UNIVERSITY, St. Louis, MO (US)

(21) Appl. No.: 12/746,682

(22) PCT Filed: Dec. 8, 2008
(86) PCT No.: PCT/US08/85784

§ 371 Date: Sep. 28, 2010

Related U.S. Application Data
(60) Provisional application No. 60/992,823, filed on Dec. 6, 2007.

(51) Int. Cl. A61K 49/00; G01N 33/53; G01N 33/566

(52) U.S. Cl. 424/9.1; 435/7.92; 436/501

(57) ABSTRACT

The present invention provides a biomarker for a central nervous system inflammatory disorder. The present invention also provides processes for detecting a central nervous system inflammatory disorder and processes for monitoring the effectiveness of a therapeutic treatment for a central nervous system inflammatory disorder.

### GOVERNMENT SUPPORT

The present invention was made, at least in part, with federal support under National Institute of Neurological Disorders and Stroke Grant NSO45607 from the National Institutes of Health. Accordingly, the United States Government may have certain rights in this invention.

### FIELD OF THE INVENTION

The present invention generally relates to inflammatory disorders of the central nervous system (CNS). In particular, the present invention provides a biomarker for CNS inflammatory disorder, as well as processes for detecting CNS inflammatory disorders and for monitoring the effectiveness of a therapeutic treatment for CNS inflammatory disorders.

### BACKGROUND OF THE INVENTION

Multiple sclerosis (MS) is an inflammatory demyelinating disease of the CNS. MS pathology is characterized by a breakdown in the blood brain barrier such that lymphocytes and macrophages infiltrate the CNS. The infiltrating lymphocytes initiate inflammatory demyelination and macrophages phagocytose myelin, leading to areas of demyelination and/or plaque formation. The chemotactic movement of these cells is directed by a variety of chemokines, including CXCL12 whose effects are mediated by its receptor, CXCR4.

MS can be difficult to diagnose because its signs and symptoms resemble those of many other medical disorders. Typically, the diagnosis of MS is based on the presence of CNS lesions that are disseminated in time and space (i.e., occur in different parts of the CNS at least three months apart), with no better explanation for the disease process. Thus, there is a need for more sensitive and accurate diagnostic indicators of MS and other CNS inflammatory disorders. Ideally, such indicators would be able to detect the inflammatory disorder early in its development. Early diagnosis is important because early treatment helps slow the progression of the disease.

### SUMMARY OF THE INVENTION

Among the various aspects of the present invention is the provision of a method for detecting a CNS inflammatory disorder in a subject. The method comprises determining the level of activated CXCR4 in the subject. The method further comprises comparing the level of activated CXCR4 to a baseline value, wherein an increase in the level of activated CXCR4 relative to the baseline value indicates that the subject has a CNS inflammatory disorder.

Another aspect of the invention comprises a method for monitoring the effectiveness of a therapeutic treatment for a CNS inflammatory disorder. The method comprises determining the level of activated CXCR4 in a subject at a first time point, and determining the level of activated CXCR4 in a subject at a second time point. The method further comprises comparing the levels of activated CXCR4 at the first and second time points, wherein a decrease in the level of activated CXCR4 between the first and second time points indicates that the therapeutic treatment is effective.

Still another aspect of the invention provides a biomarker for a CNS inflammatory disorder. The biomarker comprises the level of activated CXCR4 in a subject.

Other aspects and features of the invention are detailed below.

### DESCRIPTION OF THE FIGURES

FIG. 1 presents images of active multiple sclerosis (MS) lesions within the medulla of a MS patient. A section from the medulla of a postmortem specimen from a patient with MS stained with Luxol fast blue (LFB) reveals multiple, irregularly bordered areas of demyelination (a). The boxed area indicates the region depicted at higher magnification after staining with hematoxylin and eosin (b), LFB (c), or oil-red O (ORO) (d). Note the irregular border of demyelination (c, arrowheads) that extends into a defined region bordered abruptly by ORO+ macrophages (d, arrowheads). The demyelinated region contains a venule with intense perivascular infiltration of small lymphocytes (e) that are adjacent to foamy ORO+ macrophages (f). Magnification: 8× (a), 40× (b-d), 100× (e, f).

FIG. 2 demonstrates that CXCL12 redistribution occurs in venules within CNS tissues derived from MS patients. Endothelial cell localization (CD31, Alexa-488, green) of CXCL12 (Alexa-555, red) in arterioles and venules within post-mortem CNS tissues derived from non-MS (a, b) and MS (c, d) patients. Note lack of CD31 staining within elastin layer of arteriole wall (large arrows). CXCL12 expression is detected along the basolateral (small arrow) and lumenal (arrowhead) surfaces of venules only in MS specimens (d). Nuclei are counterstained with ToPro3 (blue). Scale bar=8 μm.

FIG. 3 illustrates that CXCL12 redistribution is specifically altered during MS. Endothelial cell localization (CD31, Alexa-488, green) of CXCL12 (Alexa-555, red) in cerebellar tissue obtained from non-MS (ALS) (a) and MS patients (b, c). Nuclei are counterstained with ToPro3 (blue), scale bar=10 μm. Quantification of fluorescence intensity acquired by confocal microscopy for CXCL12 (red stain and line) and CD31 (green stain and line) are shown for venules within non-MS (d) and MS (e, f) tissues. Double-headed arrows indicate location of line graph analysis. Three-dimensional reconstructions of microvessels stained with anti-CXCL12 (red) and anti-hCD31 (green) antibodies are shown for venules depicted in panels a-c: non-MS (g), MS (h, i). The percentage of venules with loss of CXCL12 polarity in each patient in the non-MS and MS cohorts (j) were determined by examining CD31 and CXCL12 staining patterns in approximately 5-72 venules per patient (n=6 control, 11 MS patients).

FIG. 4 illustrates that CXCL12 redistribution occurs in venules within active MS lesions regardless of extent of perivascular infiltrates. Combined analysis of all venules with >10 perivascular leukocytes within MS specimens were examined for redistributed CXCL12 expression (light gray bar) versus polarized expression (dark gray bar) (a). Combined analysis of venules with CXCL12 redistribution for venules with >10 (light gray bar) versus <10 (dark gray bar) leukocytes adjacent to CD31+ endothelium (b). Endothelial cell localization (CD31, Alexa-488, green) of CXCL12 (Alexa-555, red) reveals loss of CXCL12 polarity in a venule without perivascular infiltrates (<10 associated leukocytes) (c). Combined analysis of all venules with >15 versus <15 perivascular leukocytes for loss of perivascular CXCL12 expression (d). A CD31+ venule (Alexa-488, green) with intense perivascular infiltrates displays weak intralumenal (arrowhead) and no perivascular (arrow) expression of CXCL12 (e). Scale bar=8 μm.

FIG. 5 demonstrates that astrocytes are a source of CXCL12. Activated astrocyte localization (GFAP, Alexa488, green) (a) of CXCL12 (Alexa-555, red) (b, arrow) in the glial limitans adjacent to CXCL12expressing endothelial cells (b, arrowheads) within a section from the optic nerve from a non-MS patient. Merged image (c). Scale bar=8 μm. Endfeet of activated astrocytes (GFAP, Alexa-488, green) (d) express increased staining for CXCL12 (Alexa-555, red) (e, arrowheads) within a section from the spinal cord from an MS patient. Merged image depicts co-localization of GFAP and CXCL12 (f, arrow).

FIG. 6 illustrates that CXCR4 activation occurs within infiltrating leukocytes in active MS lesions. Cellular localization with panCXCR4 (Alexa-488, green) (a) and pS³³⁹-CXCR4 (Alexa-555, red) (b) antibodies in a section of the medulla with an active MS lesion. Note a subset of perivascular CXCR4-expressing cells (c, arrowheads) and endothelial cells (c, white arrow) contain activated CXCR4. Analyses of an inflamed CD31-positive (Alexa-488, green) venule within sister-sections derived from an active MS lesion in the midbrain reveals redistribution of CXCL12 (Alexa-555, red) (d) is associated with activation of CXCR4 (Alexa-555, red) (f, arrowheads) within CD45-positive leukocytes (Alexa-488, green) (e). The white line delineates the CD31-positive venule perimeter (d, arrow) detected in the merged image (g). Analysis of CXCR4 activation in control CNS specimens reveals CD45-positive (Alexa-488, green), pS³³⁹-CXCR4-positive (Alexa-555, red) cells within the perivascular space in a thoracic cord section from a patient with CNS lymphoma (h) but no CXCR4 activation in CD45-positive cells within the lumen of a vessel in the cerebrum of an ALS patient (i). Scale bar=20 μm.

FIG. 7 illustrates that the redistribution of CXCL12 significantly correlates with histological markers of MS disease severity. Severity scores of inflammation (a), demyelination (b), and presence of macrophages (c) within CNS sections derived from MS patients are plotted versus percentage of CXCL12 redistribution for each block analyzed in the MS patient group. Correlation coefficient of best-fit line (r²) and p-values (p) are shown for each graph.

### DETAILED DESCRIPTION OF THE INVENTION

It has been discovered that the normal basolateral localization of CXCL12 is disrupted in active lesions of patients with multiple sclerosis. This disrupted localization of CXCL12 is associated with an increase in CXCR4 activation in infiltrating leukocytes. Consequently, the leukocytes are able to migrate across the compromised blood-brain barrier and initiate the autoimmune inflammation characteristic of the disorder. Thus, the levels of activated of CXCR4 may be used as an biomarker for detecting and/or monitoring the progression of multiple sclerosis and other inflammatory disorders of the central nervous system. Furthermore, the levels of activated CXCR4 may be used to monitor the effectiveness of a therapeutic treatment for an inflammatory disorder of the central nervous system.

### I. Method for Detecting a Central Nervous System Inflammatory Disorder

One aspect of the present invention provides a method for detecting an inflammatory disorder of the central nervous system (CNS) in a subject. The method comprises measuring the level of activated CXCR4 in the subject and comparing the level of activated CXCR4 to a baseline value, wherein an increased level of activated CXCR4 relative to the baseline value indicates that the subject has a CNS inflammatory disorder. Furthermore, the magnitude of the increase in the level of activated CXCR4 relative to the baseline value is positively correlated with the severity of the CNS inflammatory disorder. The method of the invention may be used to detect, diagnose, access the severity, and/or monitor the progression of a CNS inflammatory disorder, and, in particular, to detect, diagnose, access the severity, and/or monitor the progression of multiple sclerosis.

a. Activated CXCR4

The method comprises measuring the level of activated CXCR4 in the test subject and determining whether the level is elevated relative to a baseline value, wherein an increased level indicates the presence of a CNS inflammatory disorder. CXCR4 is a heterotrimeric G-protein coupled receptor that is typically activated by the chemokine CXCL12 (also known as SDF-1). CXCL12, which induces chemotaxis in specific cells, binds to CXCR4 in the membrane of a responsive cell, leading to the activation of CXCR4 and, consequently, chemotaxis. Thus, binding of its ligand (i.e., typically CXCL12) may activate CXCR4.

In one embodiment, the activation of CXCR4 may be associated with increased levels of CXCL12. The increased levels may be due to increased synthesis or decreased degradation of CXCR4. In another embodiment, the activation of CXCR4 may be associated with altered expression of CXCL12. That is, the expression of CXCL12 may be up-regulated (e.g., at the level of transcription, RNA processing, or translation). Alternatively, CXCL12 may be expressed in ectopic locations. In still another embodiment, the activation of CXCR4 may be associated with disrupted localization of CXCL12. For example, the normal polarized localization of CXCL12 in microvessels of the CNS may be perturbed such that CXCL12 is also localized to the luminal surface of the vessels. As a consequence, CXCR4 is activated within luminal mononuclear cells, which then may migrate into the CNS and initiate an inflammatory lesion. In an alternate embodiment, CXCR4 may be activated via at least one mutation in the gene that encodes CXCR4. The mutation may result in a form of CXCR4 that is constitutively active, such that activation of CXCR4 is independent of ligand binding. The mutation may be synthetically induced or naturally occurring. In still another embodiment, the activation of CXCR4 may comprise a change in the conformation of the protein. In yet another embodiment, activated CXCR4 may comprise a change in the phosphorylation status of the protein. That is, a specific amino acid residue may be phosphorylated or dephosphorylated. In an exemplary embodiment, activated CXCR4 comprises at least one specific phosphorylated amino acid residue, whereas inactive CXCR4 lacks a phosphate group on each of these specific amino acid residues.

b. Determining the Level of Activated CXCR4

The levels of activated CXCR4 may be measured by a variety of methods, all of which are well known to those of skill in the art. Additional guidance may be found in reference manuals such as Current Protocols in Cell Biology, Bonifacino et al, Wiley, N.Y. or Ausubel et al. Current Protocols in Molecular Biology, 2003, Wiley, N.Y. In one embodiment, the level of activated CXCR4 may be measured by detecting the amount of ligand-bound CXCR4 or the amount of CXCR4 associated with a trimeric G protein or a G protein subunit. In another embodiment, the level of activated CXCR4 may be measured by distinguishing the active, phosphorylated form of CXCR4 from the inactive, non-phosphorylated form of CXCR4 by mass spectrometry, for example. In still another embodiment, the level of activated CXCR4 may be measured by using a specific antibody that recognizes activated CXCR4 but does not recognize the inactive form of CXCR4.

For example, the specific antibody may recognize ligand-bound CXCR4, but not recognize the unbound or free form of CXCR4. Alternatively, the specific antibody may recognize CXCR4 that is activated via a mutation. In yet another embodiment, the specific antibody may be a phospho-specific antibody that recognizes the active, phosphorylated form of CXCR4, but not the inactive, non-phosphorylated form of CXCR4. As an example, the phospho-specific antibody may recognize CXCR4 that has a phosphate group on serine 339, but not recognize CXCR4 that does not have a phosphate group on serine 339. Alternatively, the phospho-specific antibody may recognize CXCR4 that has a phosphate group on another amino acid residue, but not recognize CXCR4 that does not have a phosphate group on that amino acid residue. In a preferred embodiment, the specific antibody may be a phospho-specific antibody that recognizes phospho-serine 339 of CXCR4. An example of such an antibody is disclosed in patent application publication no. WO 2007/005605, which is incorporated herein by reference in its entirety.

The specific antibody that recognizes activated CXCR4 may be a polyclonal antibody, a monoclonal antibody, a chimeric antibody, a humanized antibody, a Fab fragment, a nanobody (i.e., a single chain antibody derived from camels or llamas), a recombinant single chain antibody, a recombinant antibody fragment, and a combination thereof. The specific antibody may be commercially available or the specific antibody may be generated against activated CXCR4 or a fragment thereof using techniques that are well known in the art.

For example, polyclonal antibodies may be generated in a variety of hosts, including goats, rabbits, rats, mice, humans, and others, by injection with activated CXCR4, a fragment of activated CXCR4, or a chimeric protein comprising a fragment of activated CXCR4. Depending on the host species, various adjuvants may be used to increase the immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and corynebacterium parvum are especially preferable.

Monoclonal antibodies to activated CXCR4 or fragments thereof may be prepared using a technique that provides for the production of antibody molecules by continuous cell lines in culture. These include, but are not limited to, the hybridoma technique, the human B-cell hybridoma technique, and the EBV-hybridoma technique. (See, e.g., Kohler et al., 1975, Nature 256:495-497; Kozbor et al., 1985, J. Immunol. Methods 81:3142; Cote et al., 1983, Proc. Natl. Acad. Sci. USA 80:2026-2030; and Cole et al., 1984 Mol. Cell Biol. 62:109-120.)

In addition, techniques developed for the production of chimeric antibodies, such as the splicing of mouse antibody genes to human antibody genes to obtain a molecule with appropriate antigen specificity and biological activity may be used. (See, e.g., Morrison et al., 1984, Proc. Natl. Acad. Sci. USA 81:6851-6855; Neuberger et al., 1984, Nature 312:604-608; and Takeda et al., 1985, Nature 314:452-45). Alternatively, techniques described for the production of single chain antibodies may be adapted, using methods known in the art, to produce activated CXCR4-specific single chain antibodies. Antibodies with related specificity, but of distinct idiotypic composition, may be generated by chain shuffling from random combinatorial immunoglobulin libraries. (See, e.g., Burton, 1991, Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi et al., 1989, Proc. Natl. Acad. Sci. USA 86:3833-3837; Winter et al., 1991, Nature 349:293-299.) Antibody fragments that contain specific binding sites for activated CXCR4 receptor or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse et al., 1989, Science 246:1275-1281.)

In the production of antibodies, screening for the desired antibody may be accomplished by techniques known in the art, e.g., enzyme-linked immunosorbent assays (ELISAs). Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between activated CXCR4 and its specific antibody.

In some embodiments, the specific antibody may be labeled with a detectable marker. The marker may be either non-covalently or covalently joined to the specific antibody by methods generally known in the art. Suitable detectable markers generally comprise a reporter molecule or enzyme that is capable of generating a measurable signal. By way of non-limiting example, such detectable markers include a chemiluminescent moiety, an enzymatic moiety, a fluorescent moiety, an infrared moiety, a magnetic particle, and a radioactive moiety. In other embodiments, the specific antibody may be detected by a secondary antibody, wherein the secondary antibody is labeled with a detectable marker, as detailed above.

The specific antibody that recognizes activated CXCR4 may be used in a variety of immunoassays, all of which are familiar to those of skill in the art, to measure the levels of activated CXCR4. Additional guidance may be found in reference books, such as Harlow and Lane, Antibodies: A Laboratory Manual, 1998, Cold Spring Harbor Press, Cold Spring Harbor, N.Y. Suitable immunoassays include, but are not limited to, ELISAs, “sandwich” immunoassays, competitive and non-competitive assay systems radioimmunoassays, tissue immunolocalization assays, immunofluorescence localization assays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, Western blot assays, flow cytometry analyses, dot blot assays, strip blot assays, Luminex bead technologies, and antibody microarrays. In preferred embodiments, the immunoassay may be an ELISA or a flow cytometry analysis.

The immunoassay may be performed in vitro in a sample derived from the subject. In some embodiments, the sample may be a tissue sample such as a tissue biopsy. The tissue biopsy may be a brain biopsy, a spinal cord biopsy, or a CNS microvascular biopsy. The biopsied tissue may be fixed, embedded in paraffin or plastic, and sectioned, or the biopsied tissue may be frozen and cryosectioned. Alternatively, the biopsied tissue may be processed into individual cells or an explant, or processed into a homogenate, a cell extract, or a membranous fraction. The sample may also be primary and/or transformed cell cultures derived from tissue from the subject. In other embodiments, the sample may be a bodily fluid. Non-limiting examples of bodily fluids include cerebrospinal fluid, blood, serum, plasma, saliva, pleural fluid, lymphatic fluid, milk, sputum, semen, tears, and urine. The fluid may be used “as is”, the cellular components may be isolated from the fluid, or a protein faction may be isolated from the fluid using standard techniques. For example, a sample of cerebrospinal fluid may be fractionated into individual cellular components using techniques that are well known to those with skill in the art. In preferred embodiments, the sample may be cerebrospinal fluid, blood, or a blood-derived sample.

In other embodiments, the immunoassay may be performed in vivo. For this, the activated CXCR4/antibody complex may be detected using infrared detection, radioisotope detection, magnetic detection, and the like.

The subject may be a human, a companion animal such as a cat, a dog or a horse; a research animal such as a mouse, a rat, or another rodent, a zoo animal; or a primate such as a chimpanzee, a monkey, or a gorilla. In preferred embodiments, the subject is a human.

c. Determining Whether the Level of Activated CXCR4 is Elevated

The method of the invention further comprises comparing the level of activated CXCR4 in the subject to a baseline value to determine whether the level of activated CXCR4 is elevated. Typically, the baseline value refers to the level of activated CXCR4 in a control subject or, more preferably, a population of control subjects. Within the context of this invention, a “control subject” is an individual that does not have an inflammatory disorder and, in particular, a CNS inflammatory disorder. And, as detailed in the examples, control non-MS subjects have no detectable levels of activated CXCR4 in the CNS. In contrast, control subjects may have low but detectable levels of activated CXCR4 in blood, serum, plasma, lymph or other bodily fluids. Thus, the level of activated CXCR4 in the test subject will be compared with the baseline value (i.e., the level of activated CXCR4 in the control sample), and an increase in the measured level of activated CXCR4 in the test subject compared to the baseline value indicates the presence of a CNS inflammatory disorder in the test subject. For example, the level of activated CXCR4 in the test subject may be increased 1.2-fold, 1.5-fold, 2-fold, 5-fold, 10-fold, 50-fold, or 100-fold. In general, the magnitude of the increase in the test subject positively correlates with the severity of the inflammatory disorder of the CNS. That is, the greater the increase in the level of activated CXCR4, the greater the severity of the disorder.

In an iteration of the method, the level of activated CXCR4 and the level of inactive CXCR4 may be both measured in the sample from the test subject. The levels of activated and inactive CXCR4 may be determined using a phospho-specific antibody that recognizes activated CXCR4 and an antibody that recognizes inactive or membrane-bound CXCR4, respectively. Accordingly, a ratio of active to inactive CXCR4 may be determined. The ratio of active CXCR4 to inactive CXCR4 generally will be compared to a baseline ratio of active CXCR4 to inactive CXCR4 in a control subject or a population of control subjects that do not have a CNS inflammatory disorder. As detailed above, this ratio generally will be low or close to zero in control subjects. Accordingly, an increased ratio of active CXCR4 to inactive CXCR4 in the test subject relative to the baseline ration indicates the presence of a CNS inflammatory disorder.

Those of skill in the art will appreciate that the method of the invention also may be used to monitor the progression of a CNS inflammatory disorder, and in particular a CNS inflammatory disorder that does not respond to a therapeutic treatment as detailed below.

d. CNS Inflammatory Disorders

A CNS inflammatory disorder generally refers to a non-infectious, demyelinating, disimmune disorder. CNS inflammatory disorder include, but are not limited to, acute disseminated encephalomyelitis (ADEM), cerebral autosomal dominant arteriopathy with subcortical infarcts and leukoencephalopathy (CADASIL), multiple sclerosis (MS), neuromyelitis optica, neurosarcoidosis, and transverse myelitis. In a preferred embodiment, the CNS inflammatory disorder is MS. MS may be categorized in two distinctive groups: the relapsing-remitting type and the chronic-progressive type. The chronic-progressive variety of MS may be further categorized as primary-progressive, secondary-progressive, and progressive-relapsing MS. Rare forms of MS include Devic's syndrome and Schilder's disease.

### II. Method for Monitoring the Effectiveness of a Therapeutic Treatment

Another aspect of the invention provides a method for monitoring the effectiveness of a therapeutic treatment for an inflammatory disorder of the CNS. The method comprises administering the therapeutic treatment to a subject having a CNS inflammatory disorder. The method further comprises determining the level of activated CXCR4 in the subject at a first time point and determining the level of activated CXCR4 in the same subject at a second, later time point. The final step of the method comprises comparing the levels of activated CXCR4 at the two different time points, wherein a decrease in the level of activated CXCR4 between the first and the second time point indicates that the therapeutic treatment is effective. In contrast, no change or an increase in the level of activated CXCR4 between the first and the second time point indicates that the therapeutic treatment is not effective. The therapeutic treatment may alleviate symptoms of the CNS inflammatory disorder, it may reduce the severity of the symptoms of the CNS inflammatory disorder, or it may prevent the reoccurrence of symptoms of the CNS inflammatory disorder.

In general, the therapeutic treatment comprises administration of a therapeutic agent at regular intervals over a period of time. Those of skill in the art will appreciate that the timing of the intervals and the overall period of time of treatment can and will vary, depending on the severity of the CNS inflammatory disorder and the therapeutic agent, for example. The therapeutic agent may be a corticosteroid such as prednisone, methylprednisolone, or dexamethasone; an immunosuppressive agent such as azathioprine, methotrexate, cyclosporine, cyclophosphamide, or mitoxantrone; an immunomodulatory agent such as hydroxychloroquine, pentoxyfilline, thalidomide, or immunoglobulins; a targeted therapeutic such as interferon beta-1 a, interferon beta-1b, glatiramer acetate, mitoxantrone, natalizumab, infliximab, or etanercept, or a combination thereof. In other embodiments, the therapeutic agent may be a CXCR4 receptor blocker or antagonist such as KRH-2731 (Kureha Corp., Japan), AMB-3100 and AMD-070 (AmorMED, British Columbia, Canada) and those detailed by DeClercq and Schols (Antivir. Chem. Chemother. (2001) 12(Suppl 1):19-31) and Siebert et al. (Curr. Pharm. Des. (2004) 10(17):2041-2062). The CXCR4 antagonist may provide peripheral blockage and may not cross the blood-brain barrier. In yet other embodiments, the therapeutic agent may be a CXCL12 homolog. In further embodiments, the therapeutic agent may be an antigen such as a T-cell-receptor peptide, a T-cell-receptor peptide inhibitor, or an altered or recombinant myelin basic protein (MBP) or a MBP peptide. In still other embodiments, the therapeutic agent may be a cytokine-based agent such as a caspase 1 inhibitor, a tumor necrosis factor (TNF) inhibitor, a soluble TNF receptor, an interleukin-12 inhibitor, a phosphodiesterase (PDE) type IV inhibitor, a CCR1/CXCR3 inhibitor, a CCR1 inhibitor, or a CCR5 inhibitor (as reviewed by Martino et al., 2002, The Lancet Neurology 1:499-509).

To monitor the effectiveness of the therapeutic treatment, the first time point may be prior to the initiation of the therapeutic treatment and the second time point may be after the initiation of the therapeutic treatment. Alternatively, the first and second time points may be both after the start of the therapeutic treatment, with the second time point occurring at a period of time after the first time point. The period of time between the first and second time point may range from about 1 week to several months. One of skill in the art will appreciate that the level of activated CXCR4 may be determined at additional time points during the therapeutic treatment.

The levels of activated CXCR4 may be determined in vitro or in vivo using any of the assays detailed above in section (I)(b). The assay may utilize specific antibodies that recognize activated CXCR4, as detailed above. The types of CNS inflammatory disorders that may be treated include acute disseminated encephalomyelitis (ADEM), cerebral autosomal dominant arteriopathy with subcortical infarcts and leukoencephalopathy (CADASIL), multiple sclerosis, neuromyelitis optica, neurosarcoidosis, and transverse myelitis.

### III. Biomarker for a CNS Inflammatory Disorder

A further aspect of the invention encompasses a biomarker for an inflammatory disorder of the CNS. The biomarker comprises the level of activated CXCR4 in a subject. The activated CXCR4 may be phosphorylated, and, in particular, the activated CXCR4 may be phosphorylated on serine residue 339. The level of activated CXCR4 may be determined in the subject as detailed above. In general, increased levels of activated CXCR4 are associated with the presence of a CNS inflammatory disorder.

### DEFINITIONS

To facilitate understanding of the invention, several terms are defined below.

The term “CNS inflammatory disorder” is used in its broadest sense. It refers to acute inflammatory disorders or diseases of the CNS, as well as chronic, progressive inflammatory disorders or diseases of the CNS.

As used herein, “control subject” refers to an individual who does not have an inflammatory disorder and, in particular, a CNS inflammatory disorder.

The molecule “CXCR4” (also called fusin) is a transmembrane receptor that is specific for the chemokine CXCL12 (also called SDF-1).

As used herein, the term “therapeutic treatment” refers to the act of treating a CNS inflammatory disorder, such that the symptoms of the disorder are alleviated, the severity of the symptoms are reduced, and/or the reoccurrence of the symptoms is prevented.

As various changes could be made in the above-described methods and compositions without departing from the scope of the invention, it is intended that all matter contained in the above description and the examples presented below, shall be interpreted as illustrative and not in a limiting sense.

### EXAMPLES

The following examples illustrate various iterations of the invention.

### Examples 1-5

### Pathologic Expression of CXCL12 at the Blood-Brain Barrier Correlates with the Severity of Multiple Sclerosis

### Background

The blood-brain barrier (BBB), a specialization of capillary endothelial cells and periendothelial accessory structures, greatly restricts the movement of molecules and cells into the central nervous system (CNS), establishing immune privilege. Leukocytes that traverse the microvasculature during neuroinflammatory diseases such as multiple sclerosis (MS) have been observed to congregate within a subendothelial space between the endothelial cell basement membrane and the glial limitans prior to entering the CNS parenchyma. Thus, one of the hallmark features of MS lesions includes an intense perivascular infiltration consisting of lymphocytes and macrophages.

Dysregulation of BBB function and transendothelial migration of leukocytes are essential for the development and propagation of active lesions in multiple sclerosis (MS). Evidence from animal studies indicates that polarized expression of the chemokine CXCL12 at the BBB normally prevents leukocyte extravasation into the central nervous system (CNS) and that loss of this polarity is associated with entry of autoreactive leukocytes and initiation of autoimmune inflammation. In the following examples, the expression of CXCL12 and its receptor, CXCR4, was examined in CNS tissues derived from patients with various forms of MS and from non-MS patients.

### Experimental Protocols for Examples 1-5

Subjects. The study was approved by the Human Studies Committee and Institutional Review Board of the Washington University School of Medicine. Where mandated, written consent was obtained from all participants. Post-mortem CNS tissue from two groups of patients was studied: eleven patients with clinically defined MS followed in the Washington University Multiple Sclerosis Center and seven control individuals without histories of MS (Table I). The control group consisted of three patients without any evidence of neurological disease plus two patients with inflammatory neurologic diseases (CNS lymphoma and viral encephalitis) and two patients with non inflammatory neurologic diseases (amyotropic lateral sclerosis (ALS) and Alzheimer's disease (AD)) (Table I).

Neuropatholoqical classification of multiple sclerosis subtypes. Sex, age, ethnicity, family history of MS, disease duration, MS subtype, and immunomodulatory drug (IMD) exposures were recorded for each subject. The multiple sclerosis cases selected for this study had a range of ages at death (35-88 years), ages at multiple sclerosis onset (20-59 years) and disease duration (18-35 years), reflecting the variability of the MS population. The cohort included one patient classified as relapsing-remitting (RR), seven as secondary progressive (SP), and three as primary progressive (PP). Table I summarizes the information collected on these patients.

CNS tissues. Studies were performed on CNS tissues taken at autopsy from eleven patients with MS and seven controls. All specimens were collected within 3-9 hours of death. At each autopsy, fresh CNS tissue (brain, optic nerve, brainstem and spinal cord) was examined and when MS lesions were identified by gross inspection, these were sampled. Areas of normal-appearing CNS, both white and gray matter, were also obtained, as in some cases MS lesions are not visible grossly. One half of CNS tissues were flash-frozen at the time of autopsy and stored at −80° C. until use. At the time of autopsy or later, small areas of tissue were embedded in Optimal Cooling Temperature (O.C.T.) compound. The other part of the tissue was fixed in formalin and subsequently embedded in paraffin. For control subjects, clinical histories and cause of death were available for all cases. One-three frozen tissue blocks (1 cm³) from various CNS regions including cerebrum, optic nerve, periventricular white matter, cerebellum, brainstem and spinal cord were obtained from each MS case. Control subject CNS tissues were obtained from predominantly white matter areas corresponding to those areas sampled in the MS patients. Diagnosis of MS was histologically confirmed by a neuropathologist.

Histological scoring of CNS tissues. Inflammatory cell infiltrates and demyelination were evaluated using haematoxylin-eosin (H&E) and Luxol fast blue-Periodic acid-Schiff (LFB-PAS) staining, respectively, as described by Budde et al., 2007, Magn. Reson. Med. 57:688-695. Active MS lesions were identified by the presence of variable amount of perivascular and parenchymal lymphocytic infiltrates, macrophages and demyelination. Oil red 0 (ORO) staining was performed to demonstrate neutral lipids within macrophages (indicative of myelin phagocytosis), which are named lipid-laden or foamy macrophages (Chayen and Bitensky, 1991, Practical Histochemistry, 2^{nd} ed, Wiley, Chichester). The extent of inflammation and demyelination within each section were graded using a four-point scale (negative, +, ++, +++), as previously by Haddock et al. 2006, Mult. Scler 12:386-396. Inflammation was scored based on the number of perivascular or parenchymal mononuclear infiltrating cells accordingly to the following scale: 0=no infiltrating cells; +=fewer than 10 infiltrating cells/40× magnified microscopic field; ++=10-20 infiltrating cells/40× magnified microscopic field; +++=20-40 infiltrating cells/40× magnified microscopic field; ++++=>50 infiltrating cells/40× magnified microscopic field. Demyelination was scored based on the percentage of the section that was demyelinated: negative=no demyelination; +=10-20%; ++=20-40%; +++=40-70%; ++++=70-100%. The amount of lipid-laden macrophages infiltrating the tissue was also evaluated using a four point scale: 0=no ORO positive cells; +=rare, fewer than 10 ORO positive cells/40× magnified microscopic field; ++=10-20 ORO positive cells/40× magnified microscopic field; +++=20-40 ORO positive cells/40× magnified microscopic field; ++++=>50 ORO positive cells/40× magnified microscopic field. All scores were determined by a blinded observer.

Antibodies. The following antibodies were used for immunohistochemistry: CXCL12 rabbit polyclonal antibody (Peprotech, Rocky Hills, N.J.), polyclonal CXCR4 antibodies (panCXCR4, Leinco, St Louis, Mo.), phosphoserine 339-CXCR4 specific antibodies (pS³³⁹-CXCR4; Woerner et al., 2005, Cancer Res, 65:11392-11399), monoclonal mouse anti-human-CD31 (hCD31) (a generous gift from Dr. P.J. Newman, Blood Center of Wisconsin), hamster anti-CD45 antibodies (BD Pharmingen, San Diego, Calif.), mouse antihuman glial fibrillary acidic protein (GFAP) antibody (Dako, (Germany), and normal goat and rabbit sera and IgG isotype control antibodies (Jackson ImmunoResearch, West Grove, Pa).

Immunohistochemistry and confocal microscopy. Frozen sections were permeabilized, blocked and stained as described by Klein et al., 2005, J. Virol. 79:11457-11466. Additional blocking with image-iT Fx signal enhancer (Molecular Probes, Eugene, Oreg.) solution was used according to the manufacturer's instructions. Detection of CXCR4 with panCXCR4 or pS³³⁹-CXCR4 antibodies was performed as described by Woerner et al (2005, supra). Primary antibodies (see above) were used at the following dilutions: anti-CXC12 (1:20), pS³³⁹-CXCR4 (1:150), anti-hCD31 (1 μg/mL), panCXCR4 (1 μg/mL), GFAP or CD45 (1-10 μg/mL). Primary antibodies were detected with secondary goat or donkey anti-rabbit or mouse IgG conjugated to Alexa 555 or Alexa 488 (Molecular Probes Inc.). For immunofluorescence staining, nuclei were counterstained with ToPro3. Control sections were incubated with antisera in the presence of a 100 μmol/L excess of peptide or with isotype-matched IgG. Sections were analyzed using a Zeiss LSM 510 laser scanning confocal microscope and accompanying software. Volocity image analysis software (Improvision, Waltham, Mass.) was used to generate and analyze three dimensional renderings of confocal images. Stained regions were identified by applying a classifier to exclude objects smaller than 0.1 cubic μm and pixels of intensity less than 45 (scale 0-225).

Statistical analysis. All values are expressed as mean+standard error of the mean (s.e.m.). Comparison of median values for each group using the Mann-Whitney test was used to determine the statistical significance of vessel CXCL12 redistribution within CNS tissues derived from MS versus non-MS patients and of percentages of pS³³⁹-CXCR4-positive inflammatory cells within CNS tissues derived from patients with subtypes of MS, with values of p<0.05 considered statistically significant.

### Example 1

### Multiple Sclerosis Cohort

Tissue from eighteen subjects (eleven with MS and seven without MS) were included in the study (Table I). For the MS patients, median disease duration was 19.5 years (range 10 to 35 years). The MS cohort included one patient with RRMS, seven patients with SPMS, and three patients with PPMS.

Histological characterization of the a total of seventeen MS and twelve non-MS tissue blocks revealed that the majority of MS blocks contained active lesions and these were graded for inflammation, demyelination and infiltration by lipid-laden macrophages as described above (see Table I). In contrast, the blocks from non-MS patients did not show any pathological abnormalities, except in the case of CNS lymphoma, where there were extensive areas infiltrated with neoplastic cells and ORO+lipid-laden macrophages and the case of West Nile virus (WNV) encephalitis, where there were intense perivascular infiltrates.

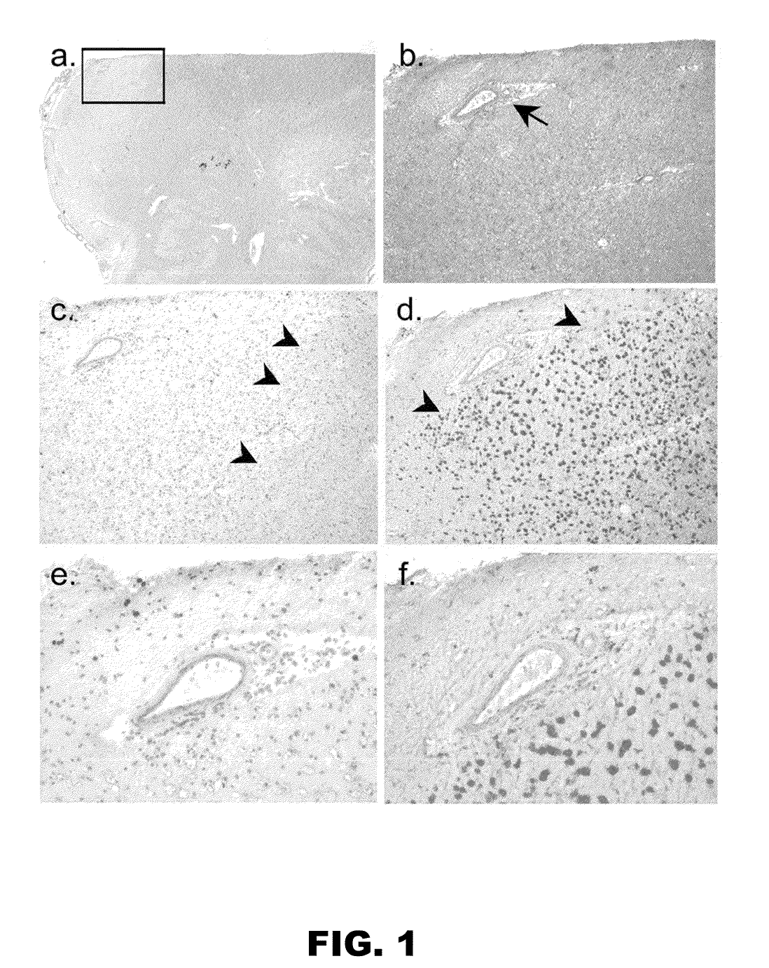

Blocks from MS patients also contained areas with normal appearing white matter, whose staining patterns were used as additional controls for CXCL12 and CXCR4 immunohistochemical analyses (see below). Demyelination in active MS lesions was associated with intense perivascular infiltrates comprised of mononuclear cells (FIG. 1a-f). Areas of extensive demyelination partially overlapped with plaque areas containing large numbers of foamy macrophages, as evidenced by staining with oil-red O (ORO) (FIG. 1c-d). Infiltrates within MS lesions consisted of mainly perivascular ORO-negative lymphocytes and parenchymal ORO-positive macrophages (FIG. 1e, f). CNS tissues derived from non-MS patients exhibited normal microvasculature without infiltrating mononuclear cells and normal myelination with the exception two patients who had CNS lymphoma and WNV encephalitis (data not shown).

TABLE 1
Patient Data

| | | | | | | |
|---|---|---|---|---|---|---|
| | age/gender/ | | | | | |
| Pt # | postmortem interval | Diagnosis | CNS level | Inflammation | Demyelination | ORO |

MS Patients

| | | | | | | |
|---|---|---|---|---|---|---|
| 01 | 46/female/6 hr | SPMS | midbrain | − | + | − |
| | | | medulla | − | − | − |
| 03 | 45/female/3.5 hr | PPMS | cerebellum | − | ++ | − |
| | | | medulla | + | + | + |
| 07 | 87/male/6 hr | SPMS | pons | − | − | − |
| 20 | 45/female/5 hr | SPMS | midbrain | ++ | + | − |
| 21 | 69/female/5 hr | PPMS | pons-plaque | ++++ | ++++ | ++++ |
| | | | 1 | | | |
| | | | pons-plaque | − | + | + |
| | | | 2 | | | |
| | | | cervical cord | ++++ | ++++ | ++++ |
| 49 | 41/female/8 hr | SPMS | lumbar cord | − | + | − |
| 50 | 35/male/10 hr | SPMS | PWM | +++ | ++ | +++ |
| | | | cervical cord | + | +++ | + |
| 59 | 55/female/8 hr | SPMS | midbrain | +++ | +++ | +++ |
| | | | cervical cord | ++++ | ++ | ++++ |
| 57 | 66/female/9 hr | RRMS | thoracic cord | − | − | − |
| 66 | 66/female/6 hr | PPMS | medulla | + | ++ | + |
| 71 | 69/male/6 hr | SPMS | cerebellum | − | − | − |

Control Patients

| | | | | | | |
|---|---|---|---|---|---|---|
| 08 | 84/female/6 hr | pulmonary | cerebellum | − | − | − |
| | | embolus | | | | |
| 32 | 56/female/4 h | emphysema | PWM | − | − | − |
| | | athero- | cervical cord | − | − | − |
| | | sclerosis | | | | |
| 38 | 39/female/4 hr | CNS | thoracic | − | ++ | ++++* |
| | | lymphoma | cord 1 | | | |
| | | | thoracic | − | ++ | ++++* |
| | | | cord 2 | | | |
| 71 | 52/male/9 hr | Congestive | optic nerve | − | − | − |
| | | heart failure | | | | |
| 65E | 80/male/5 hr | WNVE | midbrain | ++++** | − | − |
| 94 | 94 73/male/3 hr | ALS | cerebrum 1 | − | − | − |
| | | | cerebrum 2 | − | − | − |
| 95 | 34/male/3 hr | AML | thoracic cord | − | − | − |

Abbreviations:
ORO = oil red O, SPMS = secondary progressive MS, PPMS = primary progressive MS, RRMS = relapsing-remitting MS, AML = acute myelogenous leukemia, ALS = amyotropic lateral sclerosis, PWM = periventricular white matter, WNVE = West Nile virus encephalitis.
*Oil red O positive macrophages associated with tract degeneration.
**All perivascular cuff, no inflammatory cell in the parenchyma.

### Example 2

### Redistribution of CXCL12 at the BBB Occurs During MS

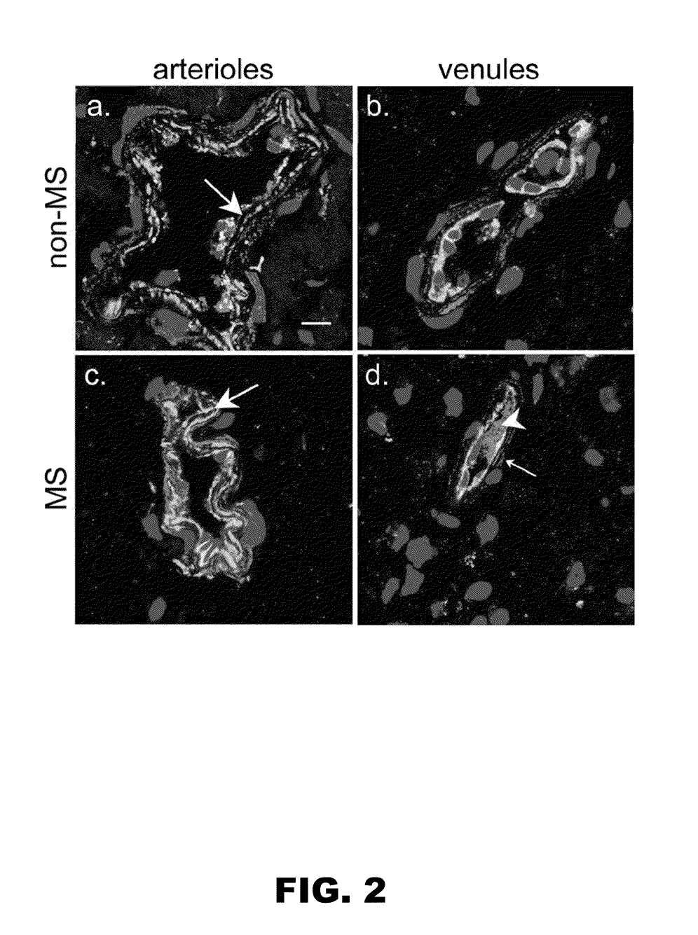

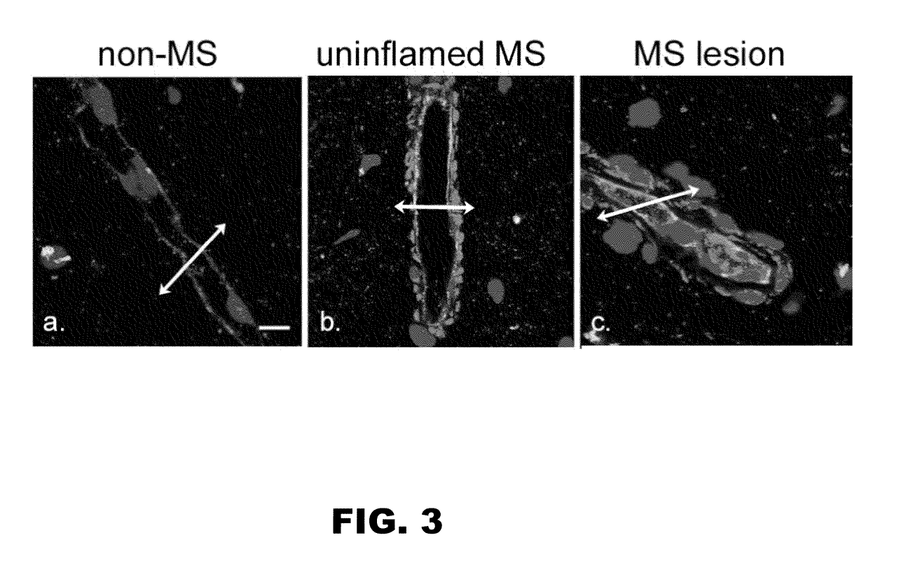

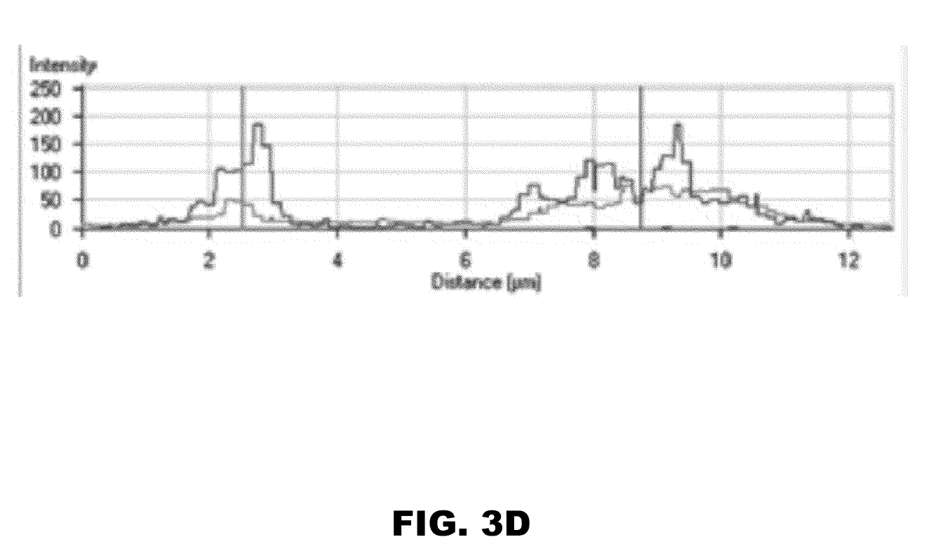

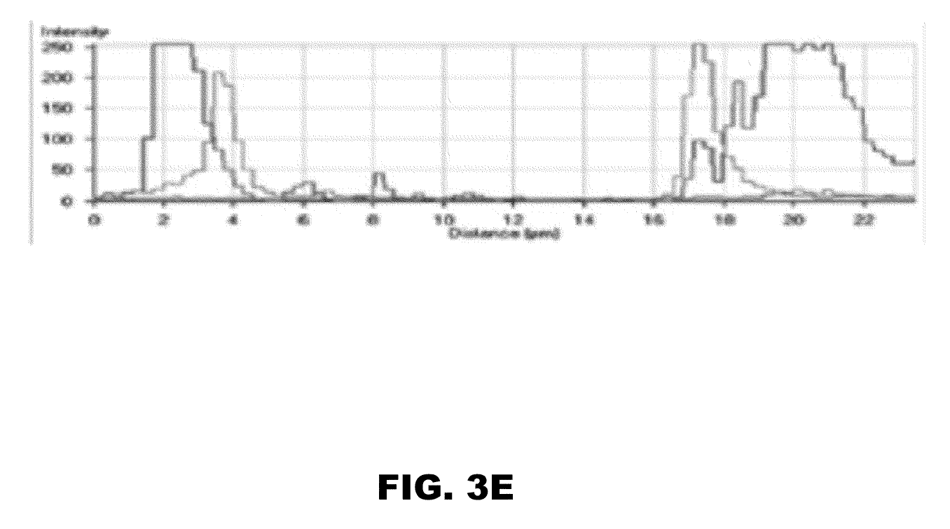

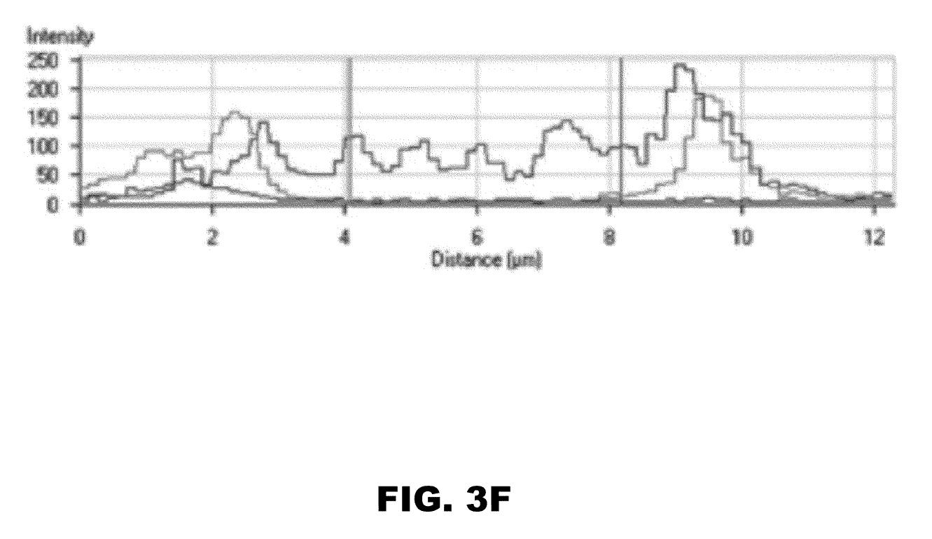

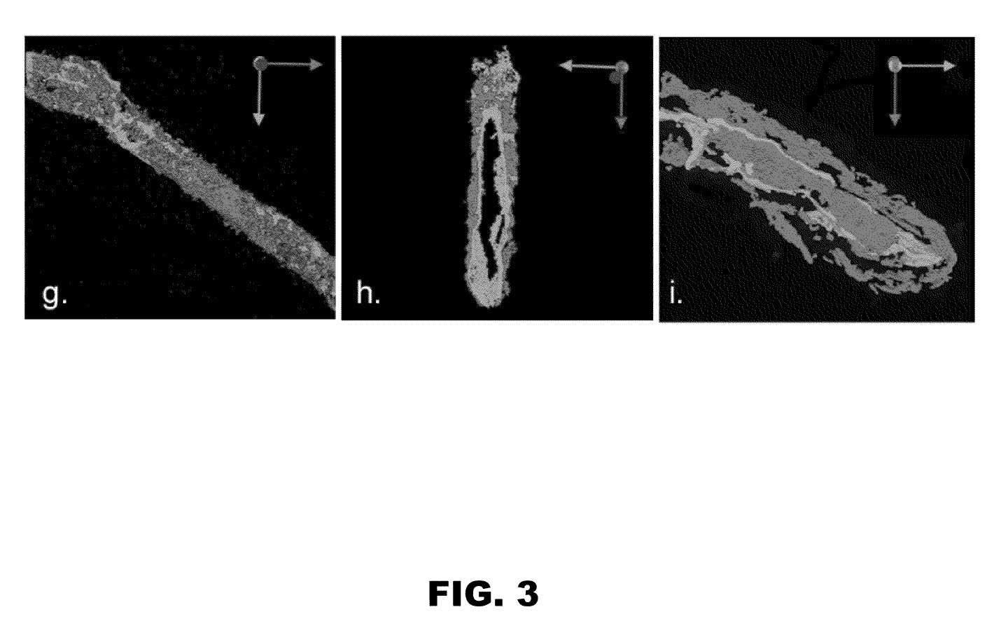

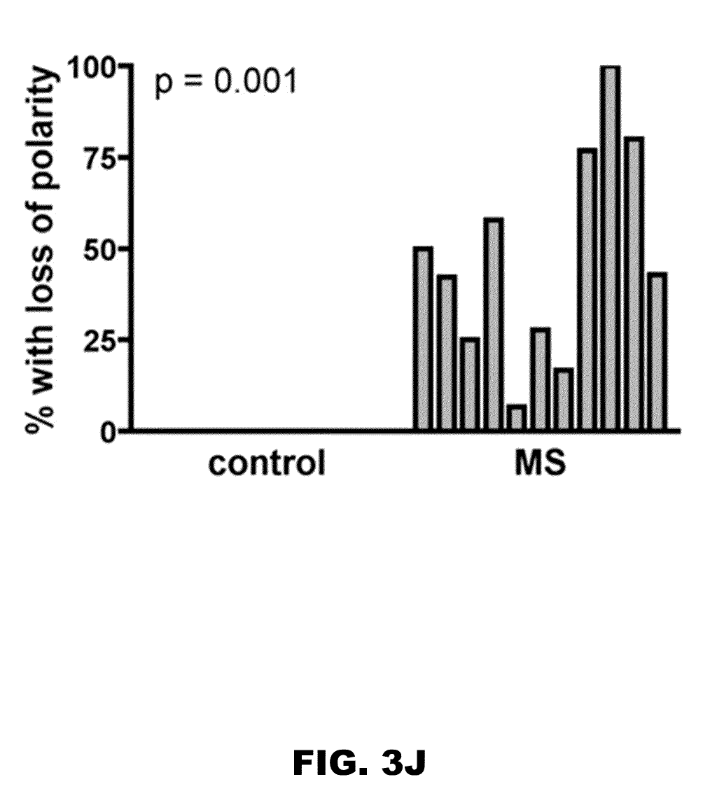

The expression CXCL12 was evaluated in all tissue blocks from MS and non-MS patients via double-label, immunofluorescent confocal microscopy. In all tissue and all CNS regions examined, CXCL12 protein was detected adjacent to staining with antibody against CD31, an endothelial cell marker, within both gray (not shown) and white matter and along both arterioles and venules (FIGS. 2a-c and 3a, b). In all venules examined within non-MS CNS tissues and in normal appearing white matter regions of those derived from MS patients, CXCL12 expression was localized to the parenchymal side of the endothelium (FIG. 3a, b). Thus, patients with histories and CNS postmortem exams consistent with CNS lymphoma, amyotropic lateral sclerosis (ALS), Alzheimer's disease (AD) or viral encephalitis did not exhibit CXCL12 redistribution to the lumenal side of the endothelium in any areas of the CNS. Interestingly, CXCL12 immunoreactivity in venules within MS specimens often displayed punctate staining, suggesting astrocyte end-feet may be a source of the chemokine (FIG. 3b). Analysis of venules within active MS lesions displayed a redistribution of CXCL12 with chemokine detected on both parenchymal and lumenal sides of CD31-expressing endothelial cells (FIGS. 2d, 3c).

In CNS tissues derived from non-MS patients and in normal appearing white matter regions of those derived from MS patients, quantification of fluorescence intensity during confocal microscopy revealed a polarity in peak endothelial cell CXCL12 expression that localized this chemokine to the basolateral surface of endothelial cells of both arterioles (data not shown) and venules (FIG. 3d, e). Quantitative confocal microscopy of inflamed venules detected a shift in the distribution of CXCL12 with respect to CD31, peak levels of intensity of CXCL12 co-localizing with peak levels of CD31 (FIG. 3f). Three-dimensional reconstructions of venules stained with CXCL12 (red) and CD31 (green) best demonstrate the redistribution of CXCL12 observed in venules within active lesions of MS tissues with non-inflamed venules exhibiting red exteriors and green interiors (FIG. 3g, h) and inflamed venules exhibiting green exteriors and red interiors (FIG. 3i). Experiments utilizing control IgG antibodies did not demonstrate any specific staining (data not shown).

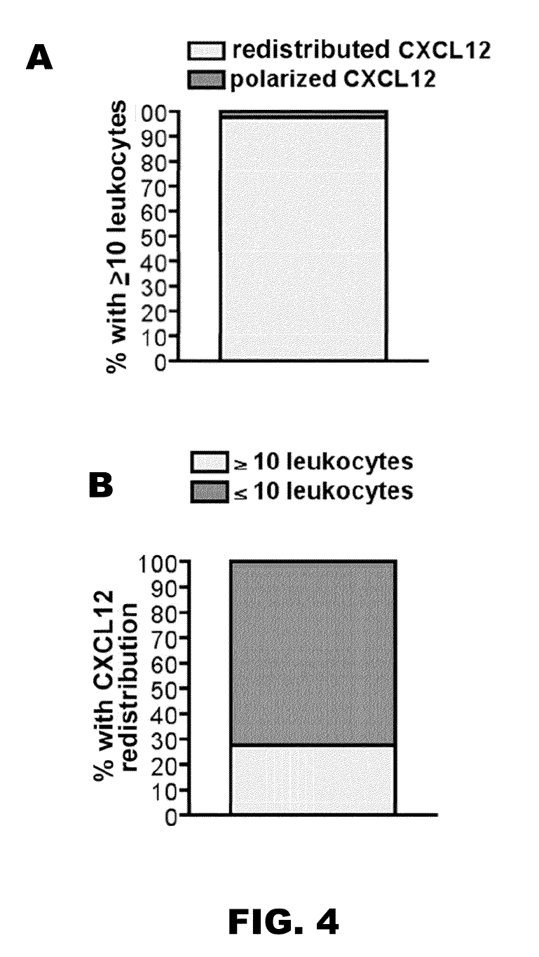

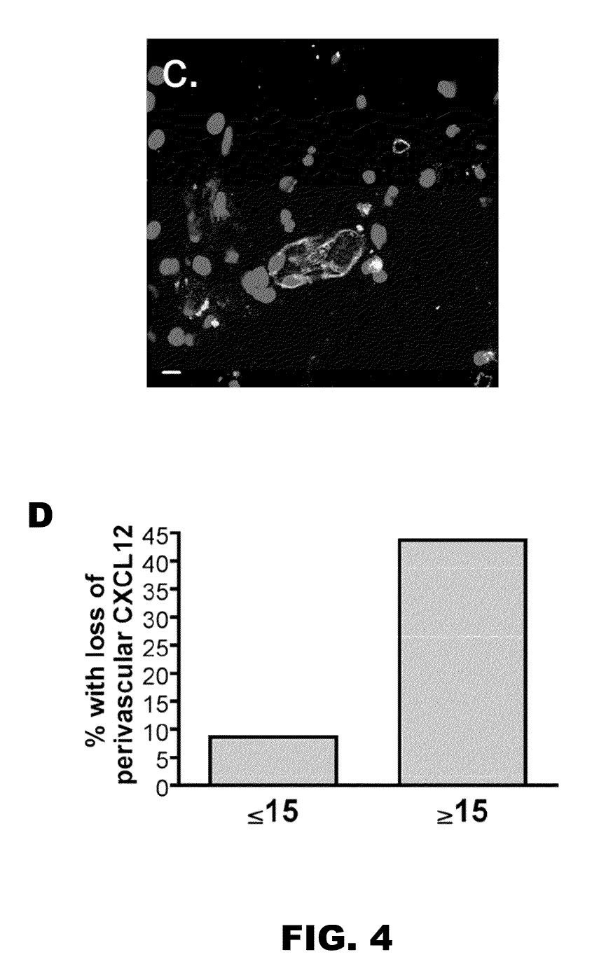

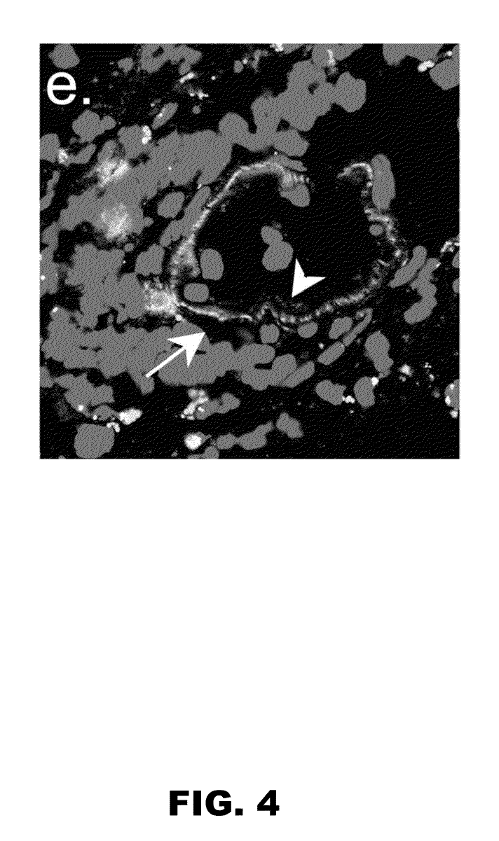

Quantitative analyses of CXCL12 expression patterns revealed that average percentages of venules displaying loss of CXCL12 polarity ranged from 10-100% in CNS sections from MS patients whereas CXCL12 remained polarized within all venules in CNS sections of non-MS patients (FIG. 3j). In general, the maximum number of venules analyzed per patient varied with the quality of the tissue available and ranged from 5-72 venules per patient. In order to determine whether CXCL12 redistribution correlated with the presence of perivascular infiltrates within MS lesions, CXCL12 expression patterns were evaluated in venules in which there were >10 leukocytes in contact with the endothelial cells. This criterion was based on assessment of un-inflamed, control specimens in which all venules had <10 leukocytes in perivascular locations (data not shown). Approximately 97% of venules meeting this criterion exhibited redistribution of CXCL12 (FIG. 4a). However, analyses of all venules with redistributed CXCL12 by this same criterion demonstrated that 72.2% had <10 perivascular leukocytes while 27.8% had >10 perivascular leukocytes (FIG. 4b-c), suggesting that CXCL12 redistribution may be detected in venules with minimal perivascular infiltrates. Examination of CXCL12 expression within venules with extensive perivascular infiltrates (>15 adjacent cells) exhibited complete loss of CXCL12 on the parenchymal side of 43.8% of venules compared with 8.7% of vessels with fewer perivacularly located cells (<15 adjacent cells) (FIG. 4d, e). These results suggest that CXCL12 is normally expressed within the perivascular spaces of arterioles and venules and disruption in this pattern of expression occurs exclusively within active lesions of MS patients. In addition, the data suggest that CXCL12 normally functions to localize leukocytes to the perivascular spaces of the CNS microvasculature and that redistribution of CXCL12 to the lumenal side of venules during MS may lead to increased parenchymal entry of infiltrating cells.

### Example 3

### Astrocyte Up-Regulation of CXCL12 Occurs at the BBB within Active MS Lesions

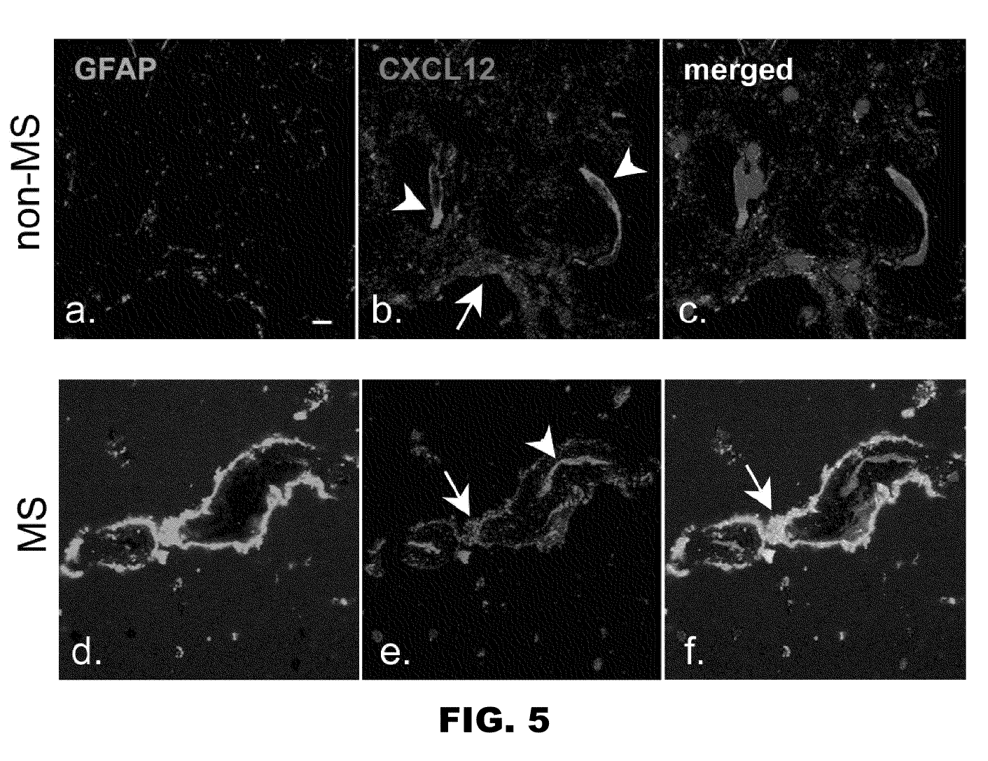

The alteration in the pattern of CXCL12 expression within inflammatory MS lesions suggested that the cellular sources of CXCL12 might differ between normal and inflamed BBB. Given that CXCL12 has been detected within astrocytes both in vitro and in vivo, CXCL12 expression was examined via confocal microscopy using antibodies to glial fibrillary acidic protein (GFAP), a marker for activated astrocytes in CNS tissues from MS and non-MS patients. In all tissues examined, microvasculature-associated astrocytes were observed to express CXCL12 (FIG. 5). In CNS sections from non-MS patients, endothelial cells expressed higher levels of CXCL12 than astrocytes (FIG. 5a-c) whereas in CNS sections from MS patients, CXCL12 expression by endothelial cells and astrocytes was comparable and the redistributed CXCL12 remained distinct from GFAP immunoreactivity (FIG. 5e). Within the MS specimens, CXCL12 and GFAP immunoreactivity were extensively co-localized within the glial limitans (FIG. 5d-f). These data suggest that astrocytes contribute to the increased levels of CXCL12 observed at the BBB within the CNS of patients with MS.

### Example 4

### CXCR4 Activation within Active MS Lesions

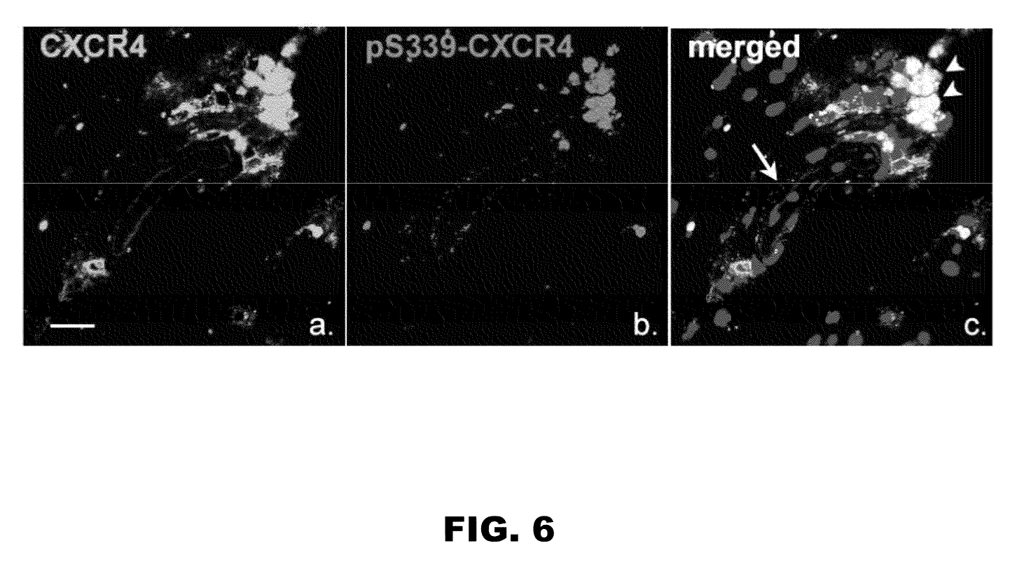

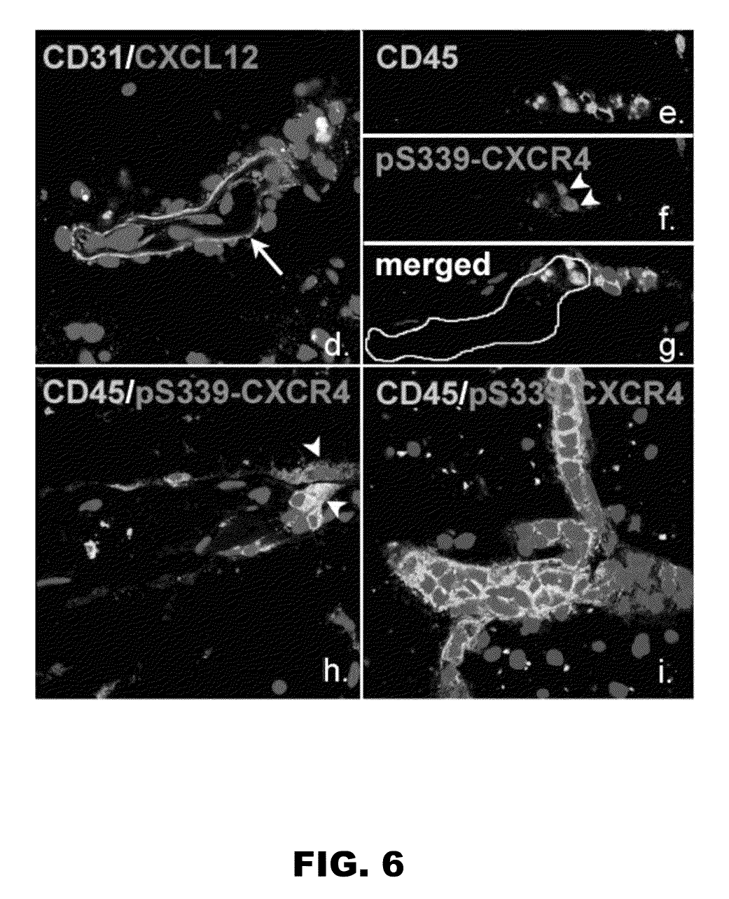

Given that the expression pattern of CXCL12 at the BBB was altered within the CNS of MS patients, the expression patterns of its receptor, CXCR4, were examined within these tissues using both panCXCR4 and pS³³⁹-CXCR4 antibodies. The latter antibody recognizes a ligand-induced phosphorylation of CXCR4 serine 339. PanCXCR4 antibodies detected CXCR4 in a majority of cells within perivascular infiltrates of active MS lesions and in scattered cells within the parenchyma (FIG. 6a). In five of the MS specimens, subsets of cells within these infiltrates exhibited ligand activated CXCR4 (FIG. 6a, b), as did the adjacent endothelial cells (FIG. 6b, c). In addition, lumenal CD45-expressing leukocytes within venules exhibiting CXCL12 redistribution also contained activated CXCR4 (FIG. 6d-g). The MS specimens in which ligand activated CXCR4 could be detected were characteristically from those with the shortest postmortem interval versus those in which no staining with pS³³⁹-CXCR4 antibodies was apparent (Table I). Decreased staining with phospho-specific antibodies due to postmortem delays has been previously observed (Ferrer et al., 2002, Acta Neuropathol. (Berl) 104:658-664).

In contrast, only one non-MS patient specimen, obtained from a patient who succumbed to CNS lymphoma, contained leukocytes with activated CXCR4, which were located in the perivascular space (FIG. 6h). In all other control specimens, CD45-expressing cells detected within the vessel lumens did not exhibit activated CXCR4 (FIG. 6h, i and data not shown), although some of these specimens had postmortem intervals in which pS³³⁹-CXCR4 would be detectable (Table I). These data suggest that the redistribution of CXCL12 in active MS lesions induces CXCR4 activation within lumenal mononuclear cells, which could lead to inappropriate trafficking of these cells into the CNS and the development of inflammatory lesions.

### Example 5

### Loss of CXCL12 Polarity at the BBB Correlates with Severity of MS

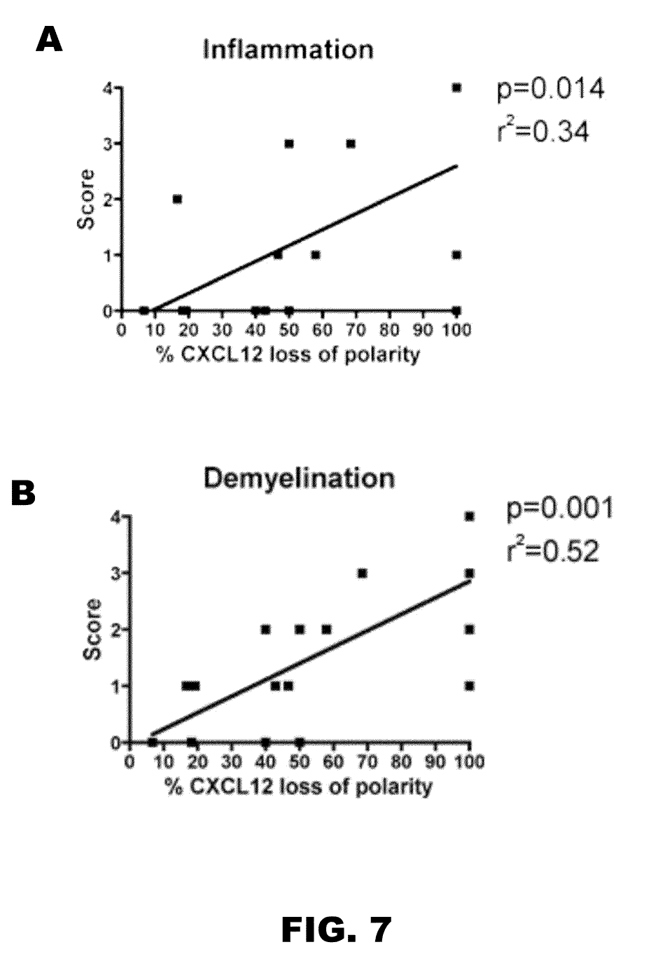

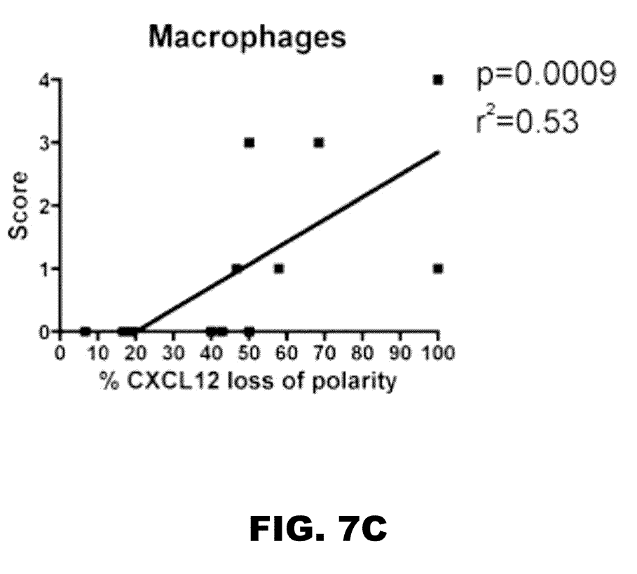

Given the association between CXCL12 redistribution and CXCR4 activation, it was hypothesized that altered CXCL12 expression within MS lesions might correlate with severity of disease. Thus, correlation analyses were performed to compare the percentages of vessels with CXCL12 redistribution with extent of inflammation, demyelination and macrophage infiltration observed within tissues derived from MS patients (Table I) (FIG. 7). While CXCL12 redistribution was significantly correlated with all measures of histological severity, the correlation coefficients for demyelination and macrophage infiltration were higher, suggesting that CXCL12 redistribution is a sensitive measure of disease severity. These data also suggest that the redistribution of CXCL12 is pathological and may play a role in ongoing capture of CXCR4-expressing leukocytes at the BBB, leading to the development of active MS lesions and associated demyelination.

1. A method for detecting a central nervous system (CNS) inflammatory disorder in a subject, the method comprising:
a. determining the level of activated CXCR4 in the subject; and
b. comparing the level of activated CXCR4 to a baseline value, wherein an increase in the level of activated CXCR4 relative to the baseline value indicates that the subject has a CNS inflammatory disorder.

2. (canceled)

3. The method of claim 1, wherein CXCR4 is phosphorylated on serine residue 339.

4. The method of claim 3, wherein phosphorylated CXCR4 is detected by a specific antibody that recognizes and binds to CXCR4 when CXCR4 has a phosphate group on serine residue 339, but not when CXCR4 does not have a phosphate group on serine residue 339.

5. (canceled)

6. (canceled)

7. The method of claim 4, wherein the level of activated CXCR4 is determined using an in vitro assay selected from the group consisting of an enzyme-linked immunosorbent assay, a flow cytometry analysis, a dot blot assay, a Western blot assay, and an immunohistochemical localization assay.

8. The method of claim 7, wherein the assay is performed with a sample selected from the group consisting of cerebrospinal fluid, blood, plasma, serum, lymph, and CNS tissue.

9. The method of claim 4, wherein the level of activated CXCR4 is determined in viva

10. (canceled)

11. The method of claim 1, wherein the baseline value is the level of activated CXCR4 in a population of control subjects.

12. The method of claim 1, wherein the magnitude of the increase in the level of activated CXCR4 is positively correlated with the severity of the CNS inflammatory disorder.

13. The method of claim 1, wherein the CNS inflammatory disorder is selected from the group consisting of acute demyelinating encephalomyelitis (ADEM), cerebral autosomal dominant arteriopathy with subcortical infarcts and leukoencephalopathy (CADASIL), multiple sclerosis, neuromyelitis optica, neurosarcoidosis, and transverse myelitis.

14. The method of claim 1, wherein the CNS inflammatory disorder is multiple sclerosis.

15. A method for monitoring the effectiveness of a therapeutic treatment for a central nervous system (CNS) inflammatory disorder, the method comprising:
c. administering the therapeutic treatment to a subject in need thereof;
d. determining the level of activated CXCR4 in the subject at a first time point;
e. determining the level of activated CXCR4 in the subject at a second time point;
f. comparing the levels of activated CXCR4 at the first and second time points, wherein a decrease in the level of activated CXCR4 between the first and second time points indicates that the therapeutic treatment is effective.

16. The method of claim 15, wherein step (b) occurs before step (a).

17. (canceled)

18. The method of claim 16, wherein CXCR4 is phosphorylated on serine residue 339.

19. The method of claim 18, wherein phosphorylated CXCR4 is detected by a specific antibody that recognizes and binds to CXCR4 when CXCR4 has a phosphate group on serine residue 339, but not when CXCR4 does not have a phosphate group on serine residue 339.

20. (canceled)

21. (canceled)

22. The method of claim 19, wherein the level of activated CXCR4 is determined using an in vitro assay selected from the group consisting of an enzyme-linked immunosorbent assay, a flow cytometry analysis, a dot blot assay, a Western blot assay, and an immunohistochemical localization assay.

23. The method of claim 22, wherein the assay is performed with a sample selected from the group consisting of cerebrospinal fluid, blood, plasma, serum, lymph, nervous tissue, and CNS tissue.

24. The method of claim 19, wherein the level of activated CXCR4 is determined in vivo.

25. (canceled)

26. The method of claim 15, wherein the CNS inflammatory disorder is selected from the group consisting of acute demyelinating encephalomyelitis (ADEM), cerebral autosomal dominant arteriopathy with subcortical infarcts and leukoencephalopathy (CADASIL), multiple sclerosis, neuromyelitis optica, neurosarcoidosis, and transverse myelitis.

27. The method of claim 15, wherein the CNS inflammatory disorder is multiple sclerosis.

28. The method of claim 15, wherein the therapeutic treatment comprises administration of a therapeutic agent at regular intervals over a period of time, the therapeutic agent being selected from the group consisting of a corticosteroid, an immunosuppressive agent, an immunomodulatory agent, and a targeted therapeutic agent.

29. The method of claim 28, wherein the targeted therapeutic agent is selected from the group consisting of interferon beta-1a, interferon beta-1b, glatiramer acetate, mitoxantrone, natalizumab, infliximab, etanercept, a CXCR4 antagonist, and combinations thereof.

30. A biomarker for a central nervous system (CNS) inflammatory disorder, the biomarker comprising the level of activated CXCR4 in a subject.

31. (canceled)

32. (canceled)

33. (canceled)

34. (canceled)
